(19) United States
(12) Patent Application Publication
(10) Pub. No.: US 2026/0102385 A1
(43) Pub. Date: Apr. 16, 2026

(54) NEW USE OF RECA INHIBITOR AND DERIVATIVE COMPOUNDS FOR TREATING DNA-RELATED DISEASES

(71) Applicant: United Arab Emirates University, Al Ain (AE)

(72) Inventors: Ahmed H. AL MARZOOQI, Al Ain (AE); Alya A. ARABI, Al Ain (AE); Jisha CHALISSERY, Al Ain (AE); Amira BEKDASH, Al Ain (AE); Tiyaba FURQAN, Al Ain (AE)

(21) Appl. No.: 18/913,657

(22) Filed: Oct. 11, 2024

(51) Int. Cl. A61K 31/4545

(52) U.S. Cl. CPC A61K 31/4545

(57) ABSTRACT

The present invention provides a method of treating or preventing various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity, related to bacterial, parasitic, viral infections/infestations or neurodegenerative, genetic, and immune disorders by administering to the subject in need thereof a compound of Formula (I) or (II), which binds to DNA affecting the biological processes that involve DNA such as chromatin remodelling functions.

### FIELD OF THE INVENTION

The present disclosure relates to methods for treating DNA-related diseases. In particular, the compounds of Formula (I) or (II) are employed for the treatment or prevention of a for gene-related disease, disorder or condition, including, but not limited to, processes that are affected as a result of binding of Formula (1) or (II) to DNA include DNA replication, transcription, recombination, DNA repair, growth and development, cell differentiation, epigenetic regulation, regulation of DNA methylation, methyl transferase enzyme modulation, topoisomerase enzyme modulation, inhibition of restriction digestion of DNA, inhibiting DNA dependent ATPase activity, inhibiting chromatin remodeling activity, or a combination thereof, in organisms including bacteria, parasites, and viruses.

### BACKGROUND OF THE INVENTION

DNA-binding compounds have been extensively utilized as antimicrobial and antiparasitic agents and for treating autoimmune diseases. Notable examples include antiprotozoal drugs like pentamidine and berenil, which bind to the minor groove of DNA[1-3]. However, pentamidine has limitations such as an inability to cross the blood-brain barrier and significant side effects including nephrotoxicity, cardiotoxicity, and hepatotoxicity.

A study on mechanisms of action of hydroxychloroquine and chloroquine, demonstrated that the drug hydroxychloroquine can bind to the minor groove of double-stranded DNA and modulate production of pro-inflammatory cytokine, playing a critical role in controlling autoimmune disorders [4]. However, chloroquine and hydroxychloroquine are associated with adverse effects like myopathy, cardiomyopathy, and ocular toxicity.

Recent advances in molecular biology have underscored the critical role of DNA-binding proteins, particularly those involved in chromatin remodeling. Chromatin remodelers are essential for the regulation of DNA accessibility, which in turn affects key biological processes such as transcription, replication, recombination, and DNA repair. Dysfunction in chromatin remodeling has been implicated in a wide array of diseases, including neurodevelopmental disorders, and neurodegenerative diseases like Alzheimer's and Huntington's [5].

Each family consists of multiple subfamilies, which belong to different organisms from the simplest eukaryote (yeast) to the most complex (mammalians), sharing significant amino acid homology and 3-D structure. For instance, the SWI/SNF family in yeast includes RSC (Remodel the Structure of Chromatin) and SWI/SNF complexes, while in mammals, it includes BAF (canonical BAF or cBAF), PBAF (Polybromo-associated BAF), and GBAF (non-canonical BAF). In addition, they have similar functions which is to use the energy from ATP hydrolysis to remodel the structure of chromatin in order to make DNA more accessible. The ATPase domain is in the catalytic subunit that hydrolyses ATP. The catalytic subunit Sth1 in the RSC complex in yeast is homologous to the catalytic subunit BRM (SMARCA2/4) in SWI/SNF in mammals. There is a 3-D structure similarity of their ATPase domains of Sth1 and BRM (SMARCA2), with an RMSD of 1.8 Å; and a 3-D structure similarity of their ATPase domains in Sth1 and BRG1 (SMARCA4), with an RMSD of 1.7 Å. These RMSD values were evaluated using the PyMOL Molecular Visualization System (see world wide website: pymol.org/). RMSD values of less than 2 Å are within the acceptable values reported by Ramirez et al. [6, 7]. A smaller RMSD means a higher structural similarity between the two superimposed proteins. FIG. 1 clearly illustrates that the Sth1 superimposes really well with each of SMARCA2 and SMARCA4. Therefore, it is possible to test biological functions on the proteins purified from any of these organisms in vitro. The data shown in FIGS. 5 and 6 were obtained based on RSC and its catalytic subunit (Sth1).

In view of the above, it can be inferred that DNA binding agents play a significant role in controlling diseases that are associated with chromatin modulation. Thus, there is an urgent need for providing a treatment regime based on DNA binding potential of the drug molecule in order to modulate cellular and chromatin activities associated with the diseased condition.

### SUMMARY OF THE INVENTION

In accordance with the present invention, disclosed herein is a method of treating or preventing various diseases, disorders, or conditions benefiting from its DNA binding properties, in modulating DNA-dependent biological processes, affecting epigenetic regulation, preventing or treating a DNA-associated disease, condition, syndrome, or disorder, inhibiting DNA dependent ATPase activity, and/or inhibiting chromatin remodeling activity, related to bacterial, parasitic, viral infections/infestations or neurodegenerative, genetic, and immune disorders, in a subject in need thereof, comprising administering to the subject:
(a) a therapeutically effective amount of a compound of Formula (I):

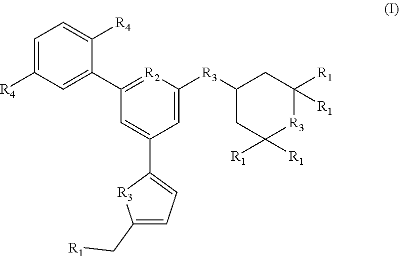

wherein R₁ is independently selected from the group consisting of:
(I) hydrogen
(II) halogen
(III) alkyl or cycloalkyl;
(IV) alkenyl or cycloalkenyl;
(V) alkynyl or cycloalkynyl;
(VI) haloalkyl;
(VII) alkoxy;
(VIII) alkylamino; or
   wherein R₂ is independently selected from the group consisting of:
(IX) a —N;
(X) a —P;
(XI) a —CR, wherein R is selected from the group comprising a hydrogen, an alkyl, a cycloalkyl, a haloalkyl, an alkylamino, or an alkoxy; or
   wherein R₃ is independently selected from the group consisting of:
(XII) an —O;
(XIII) a —S;
(XIV) a —C(R)₂, wherein R is selected from the group comprising a hydrogen, an alkyl, a cycloalkyl, a haloalkyl, an alkylamino, and/or an alkoxy;
(XV) a —NR, wherein R is selected from the group comprising a hydrogen, an alkyl, a cycloalkyl, or a haloalkyl;
(XVI) a —PR, wherein R is selected from the group comprising a hydrogen, a halogen, an alkyl, a cycloalkyl, or a haloalkyl; or
   wherein R₄ is independently selected from the group consisting of:
(XVII) hydrogen;
(XVIII) halogen
(XIX) alkyl or cycloalkyl;
(XX) alkenyl or cycloalkenyl;
(XXI) alkynyl or cycloalkynyl;
(XXII) haloalkyl;
(XXIII) aryl;
(XXIV) cyano;
(XXV) amino;
(XXVI) a —COOR, wherein R is a hydrogen or an alkyl;
(XXVII) a —OCOR, wherein R is an alkyl group;
(XXVIII) acyl;
(XXIX) sulfonyl;
(XXX) a —CON(R)₂, wherein R is selected from the group comprising a hydrogen and/or an alkyl;
(XXXI) a —CONHR, wherein R is selected from the group comprising a hydrogen or an alkyl;
(XXXII) a —PO(OR)₂, wherein R is selected from the group comprising a hydrogen and/or an alkyl;
(XXXIII) an —OR, wherein R is selected from the group comprising a hydrogen, an alkyl, a cycloalkyl, or a haloalkyl;
(XXXIV) an —N(R)₂, wherein R is selected from the group comprising a hydrogen, an alkyl, a cycloalkyl, and/or a haloalkyl;
(XXXV) a —SR, wherein R is selected from the group comprising a hydrogen, an alkyl, a cycloalkyl, or a haloalkyl;
(XXXVI) a nitro;
(XXXVII) a phosphoryl; or
   a pharmaceutically acceptable salt, co-crystal, polymorph, hydrate, solvate, prodrug, N-oxide, bioisostere, or a stereoisomer thereof, or
(b) a therapeutically effective amount of a composition comprising a therapeutically effective amount of the compound of Formula (I), or a pharmaceutically acceptable salt, co-crystal, polymorph, hydrate, solvate, prodrug, or a stereoisomer thereof.

In accordance with the present invention, in a second aspect disclosed herein is a method of a method of treating or preventing various diseases, disorders, or conditions benefiting from its DNA binding properties, in modulating DNA-dependent biological processes, affecting epigenetic regulation, preventing or treating a DNA-associated disease, condition, syndrome, or disorder, inhibiting DNA dependent ATPase activity, and/or inhibiting chromatin remodeling activity, related to bacterial, parasitic, viral infections/infestations or neurodegenerative, genetic, and immune disorders, in a subject in need thereof, comprising administering to the subject in need thereof:
(i) a therapeutically effective amount of a compound of Formula (II):

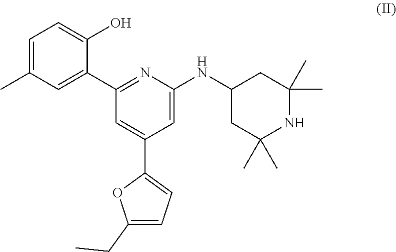

(ii) or a pharmaceutically acceptable salt, co-crystal, polymorph, hydrate, solvate, prodrug, N-oxide, bioisostere or a stereoisomer thereof, or
(ii) a composition comprising a therapeutically effective amount of the compound of Formula (II), or a pharmaceutically acceptable salt, co-crystal, polymorph, hydrate, solvate, prodrug, or a stereoisomer thereof as an active ingredient.

In a preferred embodiment, the present disclosure provides a use of a compound of Formula (I) or (II), or a pharmaceutically acceptable salt, co-crystal, polymorph, hydrate, solvate, prodrug, N-oxide, bioisostere or a stereoisomer thereof, or a composition comprising a therapeutically effective amount of the compound of Formula (I) or (II), or a pharmaceutically acceptable salt, co-crystal, polymorph, hydrate, solvate, prodrug, or a stereoisomer thereof, for the treatment of various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity, related to bacterial, parasitic, viral infections/infestations or neurodegenerative, genetic, and immune disorders.

In another aspect, the present disclosure provides a use of a compound of Formula (I) or (II) or a pharmaceutically acceptable salt, co-crystal, polymorph, hydrate, solvate, prodrug, N-oxide, bioisostere or a stereoisomer thereof, or a composition comprising a therapeutically effective amount of the compound of Formula (I) or (II), or a pharmaceutically acceptable salt, co-crystal, polymorph, hydrate, solvate, prodrug, or a stereoisomer thereof, in the manufacture of a medicament for treating various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity, related to bacterial, parasitic, viral infections/infestations or neurodegenerative, genetic, and immune disorders.

In yet another aspect, provided herein are methods of treating various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity related to bacterial, parasitic, viral infections/infestations or neurodegenerative, genetic, or immune disorders, which optionally include administering the compounds in conjunction with a therapeutic agent to a subject. A therapeutic agent comprises any treatment applicable for the treatment of an infection/infestation related to bacteria, parasites, or viruses or neurodegenerative, genetic, and immune disorders.

### BRIEF DESCRIPTION OF DRAWINGS OF THE INVENTION

FIG. 1 illustrates the superimposition of Sth1 with each of SMARCA2 and SMARCA4, (A) superimposed Sth1 (in green) with SMARCA2 (in pink), RMSD=1.8 Å, and (B) superimposed Sth1 (in green) with SMARCA4 (in pink), RMSD=1.7 Å, wherein PyMOL (see world wide website: pymol.org/) was used to do the superimposition of the structures of the ATPase domains. The ATPase domain was extracted from the following sources: Sth1 was obtained from world wide website: resb.org/structure/6vz4, the structure of SMARCA2 and SMARCA4 were obtained from AlphaFold (see world wide website: alphafold.ebi.ac.uk/) using the sequences from world wide web: uniprot.org/uniprotkb/P51531/entry for SMARCA2, and world wide web: uniprot.org/uniprotkb/P51532/entry for SMARCA4.

FIG. 2 illustrates the binding of Proflavine (A) and binding of the compound of Formula (II) (B) in between stacked base pairs separately.

FIG. 3 illustrates the binding of Chloroquine (A) and binding of the compound of Formula (II) (B) to DNA grooves separately.

FIG. 4 illustrates the DNA binding assay, Left panel (4A): Titration of Formula (II) compound binding to DNA; Right Panel (4B): SDS release of Formula (II) compound binding to DNA.

FIG. 5 illustrates the inhibitory effects of the compound of Formula (II) on the restriction digestion of DNA.

FIG. 6 illustrates the inhibitory effects of the compound of Formula (II) on ATP hydrolysis by the chromatin remodeler in the presence of DNA (ATPase activity is DNA dependent).

### DEFINITIONS

As used in this description and the accompanying claims, the following terms shall have the meanings indicated, unless the context otherwise requires:

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variation thereof) “comprising”, “comprises”, “comprise”, “consisting essentially of”, “consisting”, and “consists” can be used interchangeably.

The term “exemplary” is used herein to mean “serving as an example, instance, or illustration.” Any embodiment described herein as “exemplary” is not necessarily to be construed as preferred or advantageous over other embodiments.

The term “bioisostere” refers to a compound or group that possesses near molecular shapes and volumes, approximately the same distribution of electrons and which exhibit similar physical properties such as hydrophobicity. Bioisostereic compounds affect the same biochemically associated systems as agonist or antagonists and thereby produce biological properties that are related to each other.

The terms “stereoisomer” or “stereoisomers” refer to any enantiomers, diastereomers or geometrical isomers of the compounds of Formula (I) or (II), wherever they are chiral. When the compounds of the Formula (I) or (II) and related formulae are chiral, they can exist in racemic or in optically active form. It should be understood that the invention encompasses all stereochemical isomeric forms, including diastereomeric, enantiomeric and epimeric forms, and mixtures thereof.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the terms “comprises” or “comprising” are generally used in the sense of include, that is to say permitting the presence of one or more features or components.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as the event or circumstance where the alkyl is not substituted.

The term “pharmaceutically acceptable” means that, which is useful in preparing a pharmaceutical composition that is generally safe, non-toxic and neither biologically nor otherwise undesirable and includes that which is acceptable for veterinary as well as human pharmaceutical use. These “pharmaceutically acceptable” substances are suitable for use in interact with bodily tissues without causing excessive irritation, allergic responses, or other complications, commensurate with a reasonable benefit/risk ratio.

The term “pharmaceutically acceptable salt” refers to the salts of the compounds, that is pharmaceutically acceptable and that possesses the desired pharmacological activity of the parent compound. The pharmaceutically acceptable salts of the compounds of this disclosure include those derived from suitable inorganic and organic acids and bases. Such salts include: salts derived from inorganic bases such as Li, Na, K, Ca, Mg, Fe, Cu, Zn, Al, Mn, comprising their different oxidation states; salts of organic bases such as N,N′-diacetylethylenediamine, 2-dimethylaminoethanol, isopropylamine, morpholine, piperazine, piperidine, procaine, diethylamine, triethylamine, trimethylamine, tripropylamine, tromethamine, choline hydroxide, dicyclohexylamine, metformin, benzylamine, phenylethylamine, dialkylamine, trialkylamine, thiamine, aminopyrimidine, aminopyridine, purine, pyrimidine, spermidine, and the like; chiral bases like alkylphenylamine, glycinol, phenyl glycinol and the like, salts of natural amino acids such as glycine, alanine, valine, leucine, isoleucine, lysine, arginine, serine, threonine, phenylalanine; unnatural amino acids such as D-isomers or substituted amino acids; salts of acidic amino acids such as aspartic acid, glutamic acid; guanidine, substituted guanidine wherein the substituents are selected from nitro, amino, alkyl, alkenyl, alkynyl, ammonium or substituted ammonium salts. Salts may include acid addition salts where appropriate which are sulphates, nitrates, phosphates, perchlorates, borates, hydrohalides, acetates, tartrates, maleates, citrates, succinates, methanesulfonates, benzoates, salicylates, hydroxynaphthoates, benzenesulfonates, ascorbates, and the likes thereof.

The term “polymorph” refers to the crystallization of the same compound, which differs only in the lattice.

As used herein, the terms “prevents”, “preventing”, “prevention”, “arresting”, “blocking”, “alleviating”, “relieving”, or “inhibits” refer to a method of preventing or inhibiting the onset of a disease and/or its attendant symptoms or barring a subject from acquiring a disease. As used herein, “prevent”, “preventing”, “prevention”, “arresting”, “blocking”, “alleviating”, “relieving”, or “inhibiting” also include delaying the onset of a disease and/or its attendant symptoms and reducing a subject's risk of acquiring a disease.

The terms “solvate” or “solvates” refer to a complex in which the compounds of the Formula (II) of the present disclosure are a proportional amount of solvent molecules combined. A specific solvate in which the solvent is water is referred to as a hydrate.

The term “subject” in accordance with the present invention, includes, e.g., mammals, such as dogs, cats, horses, rats, mice, monkeys, and humans.

The terms “therapeutically effective amount” or “effective amount” refer to the quantity of the compound of Formula (I) or (II) disclosed herein that (i) treats or prevents a specific disease, disorder, or condition or syndrome; (ii) reduces, improves, or eliminates one or more symptoms associated with the disease, disorder, or syndrome; or (iii) delays the onset of one or more symptoms of the disease, disorder, condition, or syndrome described herein.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal. In an embodiment, a prodrug has less than 10 percent activity relative to the free or active drug derived or released therefrom. In an embodiment, a prodrug has less than 5 percent activity relative to the free or active drug derived or released therefrom. In an embodiment, a prodrug has less than 1 percent activity relative to the free or active drug derived or released therefrom.

The terms “—CR”, “—NR”, “—PR”, “—OR”, “—SR”, or “—C(R₂)”, “—N(R)₂”, “—COOR”, “—OCOR”, “—CON(R)₂”, “—CONHR”, or “—PO(OR)₂ refer to one or two R groups attached to a carbon, nitrogen phosphorus, oxygen, or sulfur atom, for example “—CON(R)₂” refers to two R group attached to a Nitrogen, as shown in the formula below.

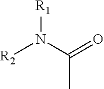

The terms “—S”, “—N”, “—O”, “—P” refer to a sulfur, nitrogen, oxygen, or phosphorus atom, and may include the forms “—S—”, “—N—”, “—O—”, “—P—” where applicable due to formula structure requirements.

The recitation of a listing of chemical groups in any definition of a variable herein includes definitions of that variable as any single group or combinations of listed groups. The recitation of an embodiment for a variable or aspect herein includes that embodiment as any single embodiment or in combination with any other embodiments or portions thereof.

Unless defined otherwise, all technical and scientific terms used herein have the same meaning as commonly understood by one of ordinary skill in the art to which the disclosure belongs. Although any methods and materials like or equivalent to those described herein may be used in the practice or testing of the present disclosure, the preferred materials and methods are described below.

### DETAILED DESCRIPTION

The present invention pertains to a method of treating or preventing various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity, related to bacterial, parasitic, viral infections/infestations, or neurodegenerative, genetic, and immune disorders in a subject in need thereof, comprising a compound of Formula (I), structurally represented as follows:
(a) a therapeutically effective amount of a compound of Formula (I):

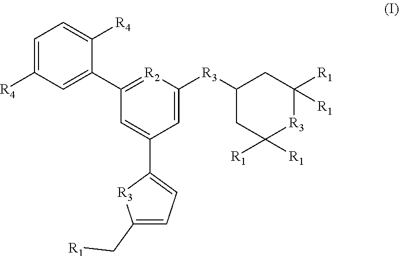

wherein R₁ is independently selected from the group consisting of:
(I) hydrogen
(II) halogen
(III) alkyl or cycloalkyl;
(IV) alkenyl or cycloalkenyl;
(V) alkynyl or cycloalkynyl;
(VI) haloalkyl;
(VII) alkoxy;
(VIII) alkylamino; or
   wherein R₂ is independently selected from the group consisting of:
(IX) a —N;
(X) a —P;
(XI) a —CR, wherein R is selected from the group comprising a hydrogen, an alkyl, a cycloalkyl, a haloalkyl, an alkylamino, or an alkoxy; or
   wherein R₃ is independently selected from the group consisting of:
(XII) an —O;
(XIII) a —S;
(XIV) a —C(R)₂, wherein R is selected from the group comprising a hydrogen, an alkyl, a cycloalkyl, a haloalkyl, an alkylamino, and/or an alkoxy;
(XV) a —NR, wherein R is selected from the group comprising a hydrogen, an alkyl, a cycloalkyl, or a haloalkyl;
(XVI) a —PR, wherein R is selected from the group comprising a hydrogen, a halogen, an alkyl, a cycloalkyl, or a haloalkyl; or
   wherein R₄ is independently selected from the group consisting of:
(XVII) hydrogen;
(XVIII) halogen
(XIX) alkyl or cycloalkyl;
(XX) alkenyl or cycloalkenyl;
(XXI) alkynyl or cycloalkynyl;
(XXII) haloalkyl;
(XXIII) aryl;
(XXIV) cyano;
(XXV) amino;
(XXVI) a —COOR, wherein R is a hydrogen or an alkyl;
(XXVII) a —OCOR, wherein R is an alkyl group;
(XXVIII) acyl;
(XXIX) sulfonyl;
(XXX) a —CON(R)₂, wherein R is selected from the group comprising a hydrogen and/or an alkyl;
(XXXI) a —CONHR, wherein R is selected from the group comprising a hydrogen or an alkyl;
(XXXII) a —PO(OR)₂, wherein R is selected from the group comprising a hydrogen and/or an alkyl;
(XXXIII) an —OR, wherein R is selected from the group comprising a hydrogen, an alkyl, a cycloalkyl, or a haloalkyl;
(XXXIV) an —N(R)₂, wherein R is selected from the group comprising a hydrogen, an alkyl, a cycloalkyl, and/or a haloalkyl;
(XXXV) a —SR, wherein R is selected from the group comprising a hydrogen, an alkyl, a cycloalkyl, or a haloalkyl;
(XXXVI) a nitro;
(XXXVII) a phosphoryl; or
   a pharmaceutically acceptable salt, co-crystal, polymorph, hydrate, solvate, prodrug, N-oxide, bioisostere, or a stereoisomer thereof, or
(b) a therapeutically effective amount of a composition comprising a therapeutically effective amount of the compound of Formula (I), or a pharmaceutically acceptable salt, co-crystal, polymorph, hydrate, solvate, prodrug, or a stereoisomer thereof.

In a preferred embodiment, the present invention pertains to a method of treating or preventing various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity, related to bacterial, parasitic, viral infections/infestations or neurodegenerative, genetic, and immune disorders in a subject in need thereof, comprising administering to the subject in need thereof a compound of Formula (II), structurally represented as follows:

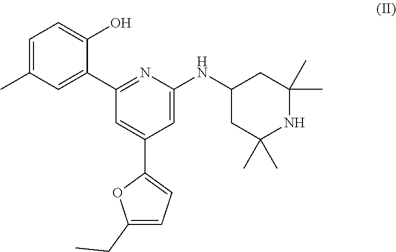

The compound of Formula (II), (2-[4-(5-ethyl-2-furanyl)-6-[(2,2,6,6-tetramethyl-4-piperidinyl)amino]-2-pyridinyl]-4-methyl-phenol), is conventionally known as a RecA inhibitor, but the use of the compound in treatment of various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity, related to bacterial, parasitic, viral infections/infestations or neurodegenerative, genetic, and immune disorders was previously unknown. In certain embodiments, the compounds of the present invention control various diseases, disorders, or conditions related to bacterial, parasitic, viral infections/infestations by inhibiting the growth and/or viability of bacteria, parasites, or viruses.

The compounds of Formula (II) may be used in the form of a free base, a salt, a co-crystal, a polymorph, a hydrate, a solvate, a prodrug, a bioisostere, or a stereoisomer thereof, including pharmaceutically acceptable forms. In certain embodiments, the compound of Formula (II) may be a pharmaceutically acceptable salt.

In certain embodiments, the present invention pertains to a method of treating or preventing various diseases, disorders, or conditions where an effective amount of the compounds of Formula (I) or (II) are administered to a subject for modulating DNA-dependent biological processes, epigenetic regulation, or preventing or treating a DNA-associated disease, condition or disorder, inhibiting DNA dependent ATPase activity, inhibiting chromatin remodeling activity, modulating DNA-dependent biological processes, affecting epigenetic regulation, regulating DNA methylation, modulating methyl transferase activities, modulating topoisomerase enzyme, or a combination thereof, where the compounds of Formula (I) or (II) bind to DNA and influence the DNA-protein binding, induce DNA damage by inhibiting repair of broken DNA strands, hinder DNA repair machinery from properly accessing and repairing damaged sites, cause DNA structural unwinding, elongation, deformations and conformational change, cause DNA strand breakage, and inhibit the relegation of broken DNA strands, or combinations thereof.

In preferred embodiments, the method of the present invention comprises administering to a subject in need thereof an effective amount of a composition comprising the compounds of Formula (I) or (II) pharmaceutically and an acceptable salt, co-crystal, polymorph, hydrate, solvate, prodrug, or a stereoisomer thereof for the treatment of cancer, microbial infection, viral infections, bacterial infections, parasitic infections, autoimmune diseases, African trypanosomiasis and Pneumocystis carinii pneumonia, intellectual disabilities and developmental disorders, Coffin-Siris Syndrome, cardiac disorders, neurodevelopmental disorder, Kleefstra syndrome, Nicolaides-Baraitser syndrome, neurodegenerative diseases, Alzheimer's disease, Huntington's disease, immunological disorders, Fragile X Syndrome, ovarian disorders, or ATR-X syndrome.

In one aspect, the present invention discloses a method of treating or preventing various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity in a subject in need thereof, comprising administering to the subject a therapeutically effective amount of a composition comprising the compound of Formula (I) or (II), or a pharmaceutically acceptable salt, co-crystal, polymorph, hydrate, solvate, prodrug, N-oxide, bioisostere or a stereoisomer thereof, and one or more excipients and/or carriers.

In another aspect, disclosed herein is the use of a compound of Formula (I) or (II) or a pharmaceutically acceptable salt, co-crystal, polymorph, hydrate, solvate, prodrug, N-oxide, bioisostere or a stereoisomer thereof, in the treatment of various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity, related to bacterial, parasitic, viral infections/infestations or neurodegenerative, genetic, and immune disorders.

In yet another aspect, the present disclosure provides a composition comprising the compound of Formula (I) or (II), or a pharmaceutically acceptable salt, co-crystal, polymorph, hydrate, solvate, prodrug, or a stereoisomer thereof, for use in the treatment of various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity, related to bacterial, parasitic, viral infections/infestations or neurodegenerative, genetic, and immune disorders.

In certain embodiments, the binding of the compounds of Formula (I) or (II) to DNA provides a therapy for various diseases where DNA binding affects biological including, but not limited to, DNA replication, transcription, recombination, and DNA repair. It also affects epigenetic regulation related to growth and development, cell differentiation, and the structure and functions of proteins, where mis-regulation can lead to a variety of diseases. In certain embodiments, the method of the present invention can be used in synthetic biology, like, for example, to switch on or off a gene. In further examples, the method of the present invention can be used for treating a disease in which one or more genes are overexpressed by switching off the one or more genes that lead to that diseases when they are overexpressed. The method of the present invention can further be used in chromatin remodeling, which play a crucial role in various cellular functions, including gene regulation, by relieving repression and promoting transcription through the remodeling of the nucleosome structure, such as, for example, nucleosome sliding, histone eviction, histone exchange, and spacing, which regulates chromatin structure at promoters and affects transcription.

In certain embodiments, the present invention provides a use of a compound of Formula (I) or (II) or a pharmaceutically acceptable salt, co-crystal, polymorph, hydrate, solvate, prodrug, N-oxide, bioisostere, or a stereoisomer thereof for modulating DNA-dependent biological processes, affecting epigenetic regulation, preventing or treating a DNA-associated disease, condition or disorder, inhibiting DNA dependent ATPase activity, inhibiting chromatin remodeling activity, regulating DNA methylation, modulating methyl transferase activities, modulating topoisomerase enzyme, or a combination thereof; wherein the compound of Formula (I) or (II) binds to DNA. In preferred embodiments, the method of the present invention provides the use of a compound of Formula (I) or (II) or a pharmaceutically acceptable salt, co-crystal, polymorph, hydrate, solvate, prodrug, N-oxide, bioisostere, or a stereoisomer thereof for preventing or treating a DNA-associated disease, condition or disorder including, but not limited to cancer, microbial infection, viral infections, bacterial infections, parasitic infections, autoimmune diseases, African trypanosomiasis and Pneumocystis carinii pneumonia, intellectual disabilities and developmental disorders, Coffin-Siris Syndrome, cardiac disorders, neurodevelopmental disorder, Kleefstra syndrome, Nicolaides-Baraitser syndrome, neurodegenerative diseases, Alzheimer's disease, Huntington's disease, immunological disorders, Fragile X Syndrome, ovarian disorders, and ATR-X syndrome.

In embodiments, the method of the present inventions includes, but is not limited to, administering to a subject in need thereof, a composition comprising the compounds of Formula (I) or (II), further comprising a pharmaceutically acceptable carrier or excipient.

In any one of the preceding embodiments, the method of the present invention comprises optionally administering an additional therapeutic agent. In certain embodiments, the additional therapeutic agent is administered prior to, concurrently with, or subsequently to the administration of the compound of Formula (I) or (II) or the composition comprising the compound of Formula (I) or (II). In another embodiment, the additional therapeutic agent is administered prior to the administration of the compound of Formula (I) or (II) or the composition comprising the compound of Formula (I) or (II). In a further embodiment, the additional therapeutic agent is administered concurrently with the administration of the compound of Formula (I) or (II) or the composition comprising the compound of Formula (I) or (II). In yet another embodiment, the additional therapeutic agent is administered subsequent to the administration of the compound of Formula (I) or (II) or the composition comprising the compound of Formula (I) or (II).

In embodiments, the method of the present invention is utilized to treat a subject in need thereof. In certain embodiments, the subject is a mammal such as a human or a non-human mammal. When administered to an animal, such as a human, the composition or the compound is preferably administered as a pharmaceutical composition comprising, for example, the compounds of the present invention and a pharmaceutically acceptable carrier. Pharmaceutically acceptable carriers are well known in the art and include, for example, aqueous solutions such as water or physiologically buffered saline or other solvents or vehicles such as glycols, glycerol, and oils such as olive oil or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The phrase “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The excipients can be chosen, for example, to effect delayed release of an agent or to selectively target one or more cells, tissues, or organs.

The pharmaceutical composition may be formulated as tablets, pills, powders, granules, capsules, suspensions, liquids for internal use, emulsions, syrups, aerosols, or sterile injectable solutions. Depending on the intended use, the composition can be administered to a subject by any of a number of routes of administration including, for example orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin or as an eye drop). The compound may also be formulated for inhalation. In yet another embodiment, the compound may be simply dissolved or suspended in sterile water.

The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be the amount of the compound which produces a therapeutic effect. Generally, out of one hundred percent, this amount will range from about 1 percent to about ninety-nine percent of active ingredients, preferably from about 5 percent to about 70 percent, most preferably from about 10 percent to about 30 percent.

Methods of preparing these formulations or compositions include the step of bringing into association an active compound, such as the compounds of the present invention with the carrier and, optionally, one or more accessory ingredients. In general, the formulations are prepared by uniformly and intimately bringing into association a compound of the present disclosure with liquid carriers or finely divided solid carriers or both and then, if necessary, shaping the product.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound and, if desired, another type of therapeutic agent being administered with the compound of the disclosure. A larger total dose can be delivered by multiple administrations of the agent. Methods to determine efficacy and dosage are known to those skilled in the art [8].

In general, a suitable daily dose of an active compound used in the compositions and methods of the disclosure will be that amount of the compound that is the lowest dose effective to produce a therapeutic effect. Such an effective dose will generally depend upon the factors described above.

As anticipated above, the compounds may be administered by any appropriate route, for example orally, parenterally, topically, or rectally. It will be appreciated that the preferred route may vary with, for example, the condition of the recipient of the compound and the condition to be treated. In certain embodiments, the extract may be especially suitable for the preparation of pharmaceuticals for intravenous administration, such as intravenous injection or infusion, provided that it does not contain components with serum-precipitating and/or haemagglutinating properties which disturb such an application. The extract may therefore be provided in the form of ampoule preparations which are directed to intravenous administration. In still other embodiments, the method comprises systemic administration of a subject composition to a subject.

Also provided are methods of treating various diseases, disorders, or conditions benefiting from its DNA binding properties, in modulating DNA-dependent biological processes, affecting epigenetic regulation, preventing or treating a DNA-associated disease, condition or disorder, inhibiting DNA dependent ATPase activity, and/or inhibiting chromatin remodeling activity, related to bacterial, parasitic, viral infections/infestations or neurodegenerative, genetic, and immune disorders, which optionally include administering the compounds in conjunction with another therapeutic agent to a subject. Conjunctive therapy includes sequential, simultaneous and separate, or co-administration of the compound and the therapeutic agent in a way that the therapeutic effect of the therapeutic agent has not entirely disappeared when the compound is administered. In certain embodiments, compound and the therapeutic agent may be compounded together in the same unitary pharmaceutical composition including both entities. Alternatively, the combination of compound and therapeutic agent may be administered separately in separate pharmaceutical compositions, each including one of the compound and one or more therapeutic agents in a sequential manner wherein, for example, the compound or the therapeutic agent is administered first and the other second. A therapeutic agent comprises any treatment applicable for the treatment of bacterial, parasitic, viral infections/infestations or neurodegenerative, genetic, and immune disorders.

Exemplary doses of the compounds in the range from about 0.001, 0.01, 0.1, 0.5, 1, 10, 15, 20, 25, 50, 100, 200, 300, 400, 500, 600, or 750 to about 1000 mg/day per kg body weight of the subject. In certain embodiments, the dose of the compound will typically be in the range of about 100 mg/day to about 1000 mg/day per kg body weight of the subject, specifically in the range of about 200 mg/day to about 750 mg/day per kg, and more specifically in the range of about 250 mg/day to about 500 mg/day per kg. In an embodiment, the dose is in the range of about 50 mg/day to about 250 mg/day per kg. In a further embodiment, the dose in the range of about 100 mg/day to about 200 mg/day per kg. In an embodiment, the dose is in the range of about 15 mg/day to 60 mg/day per kg. In a further embodiment, the dose is in the range of about 20 mg/day to 50 mg/day per kg. In an additional embodiment, the dose is in the range of about 25 mg/day to 45 mg/day per kg.

If desired, the effective daily dose of the active compound may be administered as one, two, three, four, five, six or more sub-doses administered separately at appropriate intervals throughout the day, optionally, in unit dosage forms. In yet another embodiment of the present disclosure, the active compound may be administered two or three times daily. In preferred embodiments, the active compound will be administered once daily.

The combined use of either the compound of Formula (I) or (II) with another therapeutic agents may reduce the required dosage for any individual component because the onset and duration of effect of the different components may be complementary. In such combination therapies, the different active agents may be delivered together or separately, and simultaneously or at different times within the day.

The subject or patient receiving this treatment is any animal in need, comprising primates, in particular humans. Other mammals comprise equines, cattle, swine and sheep, poultry, and pets in general.

Wetting agents, emulsifiers, and lubricants, such as sodium lauryl sulfate and magnesium stearate, as well as coloring agents, coating agents, release agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol and the like; and (3) metal-chelating agents, such as citric acid, ethylenediamine tetraacetic acid (EDTA), sorbitol, tartaric acid, phosphoric acid and the like.

In certain embodiments, the compounds of the present invention can be formulated and administered in a prodrug form. A prodrug is generally defined as a biologically inactive derivative of a parent drug molecule, which is activated by a chemical or enzymatic transformation within the body, which, in the process, involves the release of the active drug. In general, prodrugs comprise functional derivatives of the claimed compounds, which are capable of being enzymatically activated or converted into the more active parent form. Thus, in the treatment methods of the present disclosure, the term “administering” encompasses the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described [9].

The compounds of the present invention may be obtained from commercial sources or can be prepared by the conventional methods known in the art [10, 11] reveals that the BRITE compound collection, consisting of a library of more than 350,000 compounds generated by combinatorial chemistry synthetic routes, was a gift from Biogen Idec. Formula (II) was purchased from Med Chem Express.

The present disclosure now being generally described, it will be more readily understood by reference to the following examples, which are included merely for purposes of illustration of certain aspects and embodiments of the present disclosure and are not intended to limit the present disclosure in any way.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### EXAMPLES

### Example 1—Binding of a Compound of Formula (II) to DNA Compared to Binding Proflavine to DNA

The binding ability of the compound of Formula (II) to DNA either in between stacked base pairs or to DNA grooves based on computational methods was analysed. We show how compound of Formula (II) can intercalate in between DNA bases pairs, similar to how proflavine, a known intercalator, can intercalate in between the DNA base pairs [12].

Formula (II) and proflavine were docked into the same DNA structure, which is extracted from world wide web site: resb.org/structure/1n37. This DNA structure has an opening between base pairs, which is an intercalating site allowing for an intercalation of Formula (II) or proflavine in between the base pairs of DNA. Docking was performed using AutoDock Vina after having prepared the ligands and the DNA with AutoDock Tools 1.5.6. To prepare the ligands, hydrogen atoms were added, and Gasteiger charges were computed. The number of torsions in the molecule was also checked to ensure proper flexibility during docking. For the DNA preparation, hydrogen atoms were added, and Kollman charges were applied. The prepared structures were then saved in PDBQT format, suitable for docking simulations. The grid box was configured using AutoDock Tools 1.5.6, determining the dimensions and coordinates that define the search space for docking, which included the full structure of the DNA as extracted from this world wide web site: resb.org/structure/1n37. These grid parameters were then utilized in AutoDock Vina for docking the ligands with the DNA structures. The binding affinity of each ligand to DNA is reported in kcal/mol.

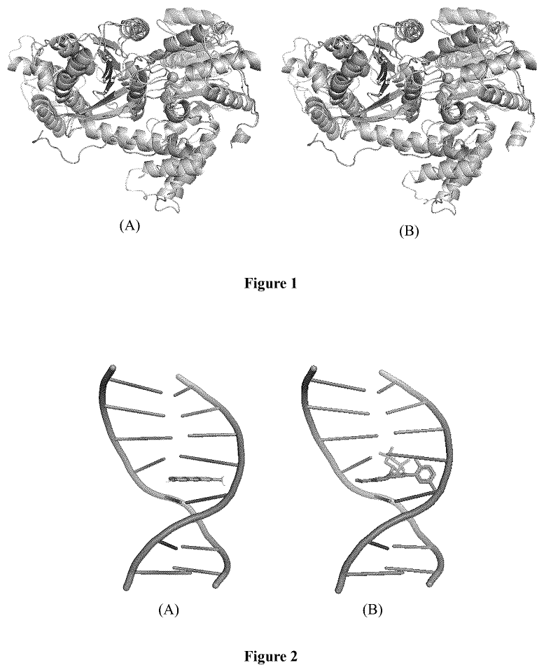

The binding of the compound of Formula (II) to DNA in between stacked based pairs has been illustrated in FIG. 2 (B). Similarly, the intercalation of proflavine in the same intercalating site is shown in FIG. 2 (A). The original ligand in the obtained DNA structure from world wide web site: resb.org/structure/1n37 was removed and both the compounds, Formula (II) and proflavine, were separately docked into this DNA structure in between base pairs as shown in FIG. 2 ((A) for Proflavine, and (B) for the compound of Formula (II)). FIGS. 2 (A) and 2 (B) shows how compound of Formula (II) intercalates in the same site as the reference compound proflavine does. This means that the two molecules can have the same biological function. The Docking score of the compound of Formula (I) was more negative with respect to the known proflavine intercalator used as a reference binder. The docking score for Proflavine was −5.8 kcal/mol, while that for Formula (II) compound was −8.2 kcal/mol. The more negative binding score of compound with Formula (II) means stronger interaction of this compound with DNA than proflavine with DNA. Thus, inferring improved binding potential of the compound of Formula (II) for DNA intercalation.

### Example 2—Binding of a Compound of Formula (II) to DNA Compared to Chloroquine

Docking analysis was performed for the compound of Formula (II) and the reference compound chloroquine, which is known to bind to DNA grooves [13, 14]. The DNA, without the opening of an intercalation site, is extracted from world wide web site: resb.org/structure/1bna. Docking was performed using AutoDock Vina after having prepared the ligands and the DNA with AutoDock Tools 1.5.6. To prepare the ligands, hydrogen atoms were added, and Gasteiger charges were computed. The number of torsions in the molecule was also checked to ensure proper flexibility during docking. For the DNA preparation, hydrogen atoms were added, and Kollman charges were applied. The prepared structures were then saved in PDBQT format, suitable for docking simulations. The grid box was configured using AutoDock Tools 1.5.6, determining the dimensions and coordinates that define the search space for docking, which included the full structure of the DNA as extracted from world wide web site: resb.org/structure/1bna. These grid parameters were then utilized in AutoDock Vina for docking the ligands with the DNA structures. The binding affinity of each ligand to DNA is reported in kcal/mol.

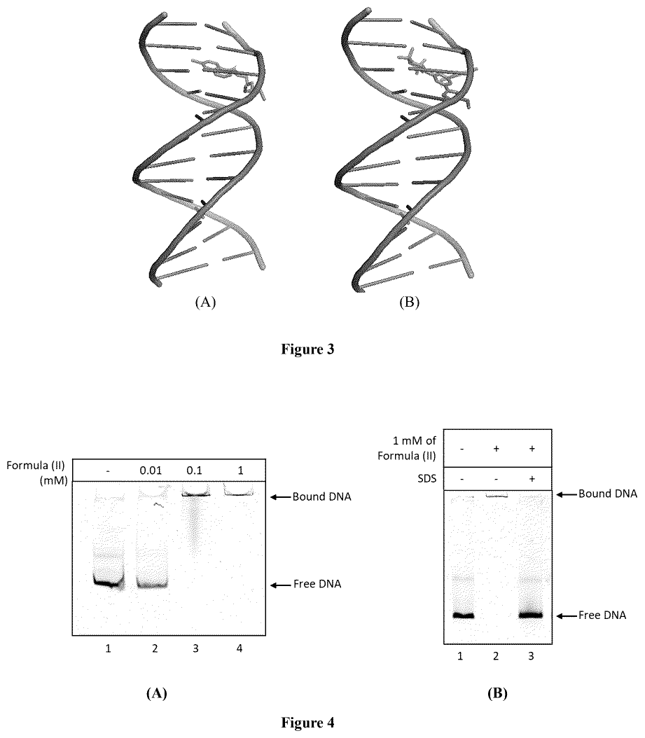

Compound of Formula (II) and Chloroquine were separately docked into DNA grooves as shown in FIG. 3 ((A) for chloroquine, and (A) for the compound of Formula (II)). FIG. 3 shows how both the compounds of Formula (II) and chloroquine bind the same site, proving that they can exert the same biological function. The Docking score of the compound of Formula (II) was comparable to that of the known chloroquine binder used as a reference binder. The docking score for chloroquine was −6.0 kcal/mol, while that for Formula (II) was −8.6 kcal/mol. The Docking score of the compound of Formula (II) is more negative compared to that of the known chloroquine DNA groove binder, which is used as a reference. The more negative binding score of Formula (II) indicates a stronger interaction of Formula (II) with DNA than that of chloroquine. Therefore, compound of Formula (II) binds, as a groove binder, stronger to DNA showing improved affinity when compared to chloroquine, used as the reference compound.

### Example 3—DNA Binding Assay

To prove the binding of Formula (II) compound to DNA experimentally, DNA binding assay for Formula (II) was carried out. For this, increasing amounts of Formula (II) compound was added to ˜20 ng of 5′-Cy5 labelled-DNA in a binding buffer that contains 10 mM Tris pH 8.0, 70 mM NaCl, 4 mM MgCl₂, 0.25 mg/ml BSA, and 1 mM DTT in the presence or absence of 0.5% SDS. After incubation for 1 h at 30° C., the samples were resolved on 8% native PAGE, and then visualized by fluorescent scanning on Sapphire biomolecular imager (Azure Biosystems). Formula (II) was found to bind efficiently to DNA (FIG. 4A lanes 3 and 4 and FIG. 4B lane 2) and the binding was dissociated in the presence of 0.5% SDS (FIG. 4B lane 3).

### Example 4—Inhibition of Restriction Digestion of DNA

To investigate whether the binding of Formula (II) to DNA, discussed in Examples 1 and 2, could lead to inhibition of restriction digestion of DNA, a restriction digestion assay method was utilized. In this assay, a 183 bp long DNA, Cy5 labelled at its 5′end, and having SalI and AccI restriction sites in the middle, was analyzed for the ability of restriction enzymes SalI and AccI to cleave DNA in the presence or absence of Formula (II). After restriction digestion for 1 h at 37° C., 5× stop buffer (100 mM EDTA, 2.5% SDS, and 1 mg/ml proteinase K) was added to the reactions, and incubated for 1 h at 50° C. Following phenol: chloroform: isoamyl alcohol extraction, the samples were resolved on 8% PAGE, and then visualized by fluorescent scanning on Sapphire biomolecular imager (Azure Biosystems). The results showed that DNA was efficiently digested by the restriction enzymes (FIG. 5 lanes 2 and 5). However, in the presence of Formula (II) compound, restriction digestion of DNA was completely inhibited as shown by the absence of a cut DNA band (FIG. 5 lanes 3 and 6). Thus, Formula (II) inhibits restriction digestion of DNA caused by two different restriction enzymes (SalI and AccI) as they are unable to access DNA in the presence of the said compound.

### Example 5—Inhibition of ATP Hydrolysis

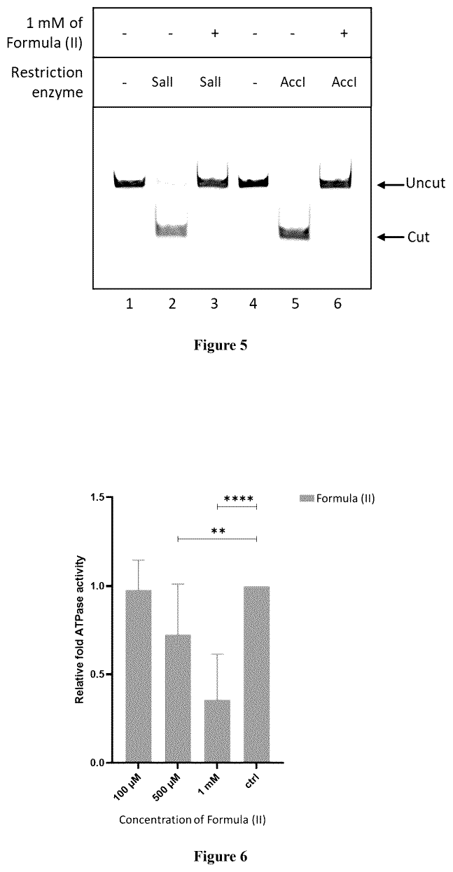

To investigate the ability of the compound of Formula (II) to inhibit the ATPase activity of RSC in the presence of DNA, ADP-Glo™ Max Assay (Promega) was used, a luminescence-based assay that detects ADP produced following ATP hydrolysis. 5 μl of reaction mixtures were prepared consisting of 5 nM RSC, 20 mM Tris pH 8, 50 mM KCl, 5 mM MgCl2, 5% glycerol, 0.1 mg/ml BSA, 1 mM DTT, 10 ng/μL salmon sperm DNA (Thermo scientific), 0.1 mM ATP, and indicated concentration of BRITE-338733. The reaction was incubated for 1 hour at 30° C. To each sample and standard, ADP-Glo Reagent and ADP-Glo Max Detection Reagent were added following manufacturer instructions. The luminescence was then measured using TECAN infinite M200 Pro. Concentration of ADP produced was calculated by correlating luminescence to standard curves prepared as per the kit protocol. Results showed that Formula (II) compound could significantly inhibit the ATPase activity of RSC in the presence of DNA. The comparison in FIG. 6 shows the results for the control vs. either 1 mM or 500 μM of Formula (II) compound used in the assay.

### REFERENCES

[1] Edwards et al., Biochemistry, 1992, 31, 7104-7109; see world wide website: pubs.acs.org/doi/abs/10.1021/bi00146a011.
[2] Brown et al., J Mol Biol, 1992, 226(2): 481-90; see world wide website: pubmed.ncbi.nlm.nih.gov/1640462/.
[3] Scott et al., European Journal of Medicinal Chemistry, 2016, 116-125; see world wide website: pure.hud.ac.uk/en/publications/an-evaluation-of-minor-groove-binders-as-anti-trypanosoma-brucei-
[4] Schrezenmeier et al., Nat Rev Rheumatol, 2020, 16(3):155-166; see world wide website: nature.com/articles/s41584-020-0372-x).
[5] Easley et al., Biochimica etBiophysicaActa (BBA)—Gene Regulatory Mechanisms, 2010, 1799 (3-4): 275-285; see world wide website: sciencedirect.com/science/article/abs/pii/S1874939909001023.
[6] Ramirez et al., Molecules, 2018; 23:1038, see world wide website: mdpi.com/1420-3049/23/5/1038.
[7] Hao et al., Sci. Rep. 2015; 15568: p. 5, see world wide website: nature.com/articles/srep15568.
[8] Wihnan, 14 Biochem. Soc. Trans. 375-82 (1986).
[9] Stella et al., Prodrugs: A Chemical Approach to Targeted Drug Delivery in Directed Drug Delivery, 247-67 (1985).
[10] Wigle et al. Journal of Biomolecular Screening, 2009, 14(9).
[11] Sexton et al., Current Chemical Genomics, 2010, 4, 34-42.
[12] Tang et al., Science, 1990, 249(4964):70-2; doi: 10.1126/science.2367853.
[13] Portilho et al., Chemical Physical Letters, 2024, 850, 141453; doi.org/10.1016/j.cplett.2024.141453.
[14] Bisoi et al., J Phys Chem B, 2023, 127(15): 3341-3351; doi: 10.1021/acs.jpcb.2c08265.

1. A method of treating a disease, condition, or disorder, the method comprising administering to a human subject in need thereof:
(a) a therapeutically effective amount of a compound of Formula (I): 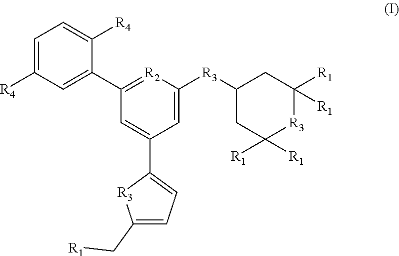
wherein R₁ is independently selected from the group consisting of:
(I) hydrogen
(II) halogen
(III) alkyl or cycloalkyl;
(IV) alkenyl or cycloalkenyl;
(V) alkynyl or cycloalkynyl;
(VI) haloalkyl;
(VII) alkoxy;
(VIII) alkylamino;
wherein R₂ is independently selected from the group consisting of:
(IX) a —N;
(X) a —P;
(XI) a —CR, wherein R is selected from the group comprising a hydrogen, an alkyl, a cycloalkyl, a haloalkyl, an alkylamino, or an alkoxy;
wherein R₃ is independently selected from the group consisting of:
(XII) an —O;
(XIII) a —S;
(XIV) a —C(R)₂, wherein R is selected from the group comprising a hydrogen, an alkyl, a cycloalkyl, a haloalkyl, an alkylamino, and/or an alkoxy;
(XV) a —NR, wherein R is selected from the group comprising a hydrogen, an alkyl, a cycloalkyl, or a haloalkyl;
(XVI) a —PR, wherein R is selected from the group comprising a hydrogen, a halogen, an alkyl, a cycloalkyl, or a haloalkyl; and
wherein R₄ is independently selected from the group consisting of:
(XVII) hydrogen;
(XVIII) halogen
(XIX) alkyl or cycloalkyl;
(XX) alkenyl or cycloalkenyl;
(XXI) alkynyl or cycloalkynyl;
(XXII) haloalkyl;
(XXIII) aryl;
(XXIV) cyano;
(XXV) amino;
(XXVI) a —COOR, wherein R is a hydrogen or an alkyl;
(XXVII) a —OCOR, wherein R is an alkyl group;
(XXVIII) acyl;
(XXIX) sulfonyl;
(XXX) a —CON(R)₂, wherein R is selected from the group comprising a hydrogen and/or an alkyl;
(XXXI) a —CONHR, wherein R is selected from the group comprising a hydrogen or an alkyl;
(XXXII) a —PO(OR)₂, wherein R is selected from the group comprising a hydrogen and/or an alkyl;
(XXXIII) an —OR, wherein R is selected from the group comprising a hydrogen, an alkyl, a cycloalkyl, or a haloalkyl;
(XXXIV) an —N(R)₂, wherein R is selected from the group comprising a hydrogen, an alkyl, a cycloalkyl, and/or a haloalkyl;
(XXXV) a —SR, wherein R is selected from the group comprising a hydrogen, an alkyl, a cycloalkyl, or a haloalkyl;
(XXXVI) a nitro;
(XXXVII) a phosphoryl; or
a pharmaceutically acceptable salt thereof, or
(b) a therapeutically effective amount of a composition comprising a therapeutically effective amount of the compound of Formula (I), or a pharmaceutically acceptable salt or polymorph thereof,
wherein the method comprises the binding of the compound of Formula (I), or a pharmaceutically acceptable salt thereof, to DNA of the human subject, and
wherein the disease, condition, or disorder is selected from the group consisting of genetic diseases, epigenetic diseases, neurodevelopmental disorders, cardiac disorders, intellectual disabilities and developmental disorders, neurodegenerative diseases, immunological disorders, immune diseases, Huntington's disease, and Fragile X Syndrome.

2. The method of claim 1, wherein the compound is of Formula (II): 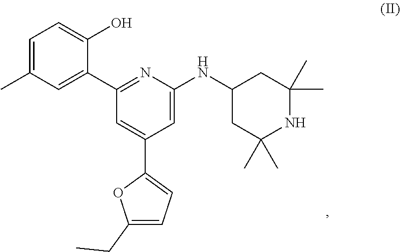
or a pharmaceutically acceptable salt thereof.

3. (canceled)

4. The method of claim 1, wherein the disease, condition or disorder is a DNA-associated disease.

5. The method of claim 1, wherein the compound of Formula (I) is used for modulating pathological cellular processes in the human subject, wherein the pathological cellular processes are selected from the group consisting of replication, transcription, recombination, DNA repair, growth and development, cell differentiation, epigenetic regulation, regulation of DNA methylation, methyl transferase enzyme modulation, topoisomerase enzyme modulation, inhibiting of restriction enzyme digestion of DNA, inhibiting DNA dependent ATPase activity, inhibiting chromatin remodeling activity, and combinations thereof.

6. The method of claim 2, wherein the compound of Formula (II) is used for modulating pathological cellular processes in the human subject, wherein the pathological cellular processes are selected from the group consisting of replication, transcription, recombination, DNA repair, growth and development, cell differentiation, epigenetic regulation, regulation of DNA methylation, methyl transferase enzyme modulation, topoisomerase enzyme modulation, inhibiting of restriction enzyme digestion of DNA, inhibiting DNA dependent ATPase activity, inhibiting chromatin remodeling activity, and combinations thereof.

7. (canceled)

8. (canceled)

9. (canceled)

10. (canceled)

11. The method of claim 1, wherein the compound is administered with one or more pharmaceutical excipients and/or carriers.

12. The method of claim 1, comprising administering one or more additional therapeutic agents, wherein the one or more additional therapeutic agents are administered prior to, concurrently with, or subsequently to the administration of the compounds of Formula (I) or the composition comprising the compound of Formula (I).

13. The method of claim 2, comprising administering one or more additional therapeutic agents, wherein the one or more additional therapeutic agents are administered prior to, concurrently with, or subsequently to the administration of the compound of Formula (II) or the composition comprising the compound of Formula (II).

14. The method of claim 1, wherein the disease, condition, or disorder is selected from the group consisting of genetic diseases, epigenetic diseases, neurodevelopmental disorders, and immunological disorders.

15. (canceled)

16. A method of modulating pathological cellular processes in a human subject, the method comprising administering to the human subject in need thereof:
(a) a therapeutically effective amount of a compound of Formula (I): 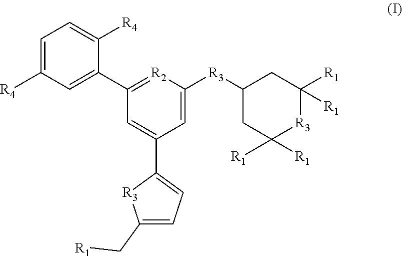
wherein R₁ is independently selected from the group consisting of:
(I) hydrogen
(II) halogen
(III) alkyl or cycloalkyl;
(IV) alkenyl or cycloalkenyl;
(V) alkynyl or cycloalkynyl;
(VI) haloalkyl;
(VII) alkoxy;
(VIII) alkylamino;
wherein R₂ is independently selected from the group consisting of:
(IX) a —N;
(X) a —P;
(XI) a —CR, wherein R is selected from the group comprising a hydrogen, an alkyl, a cycloalkyl, a haloalkyl, an alkylamino, or an alkoxy;
wherein R₃ is independently selected from the group consisting of:
(XII) an —O;
(XIII) a —S;
(XIV) a —C(R)₂, wherein R is selected from the group comprising a hydrogen, an alkyl, a cycloalkyl, a haloalkyl, an alkylamino, and/or an alkoxy;
(XV) a —NR, wherein R is selected from the group comprising a hydrogen, an alkyl, a cycloalkyl, or a haloalkyl;
(XVI) a —PR, wherein R is selected from the group comprising a hydrogen, a halogen, an alkyl, a cycloalkyl, or a haloalkyl; and
wherein R₄ is independently selected from the group consisting of:
(XVII) hydrogen;
(XVIII) halogen
(XIX) alkyl or cycloalkyl;
(XX) alkenyl or cycloalkenyl;
(XXI) alkynyl or cycloalkynyl;
(XXII) haloalkyl;
(XXIII) aryl;
(XXIV) cyano;
(XXV) amino;
(XXVI) a —COOR, wherein R is a hydrogen or an alkyl;
(XXVII) a —OCOR, wherein R is an alkyl group;
(XXVIII) acyl;
(XXIX) sulfonyl;
(XXX) a —CON(R)₂, wherein R is selected from the group comprising a hydrogen and/or an alkyl;
(XXXI) a —CONHR, wherein R is selected from the group comprising a hydrogen or an alkyl;
(XXXII) a —PO(OR)₂, wherein R is selected from the group comprising a hydrogen and/or an alkyl;
(XXXIII) an —OR, wherein R is selected from the group comprising a hydrogen, an alkyl, a cycloalkyl, or a haloalkyl;
(XXXIV) an —N(R)₂, wherein R is selected from the group comprising a hydrogen, an alkyl, a cycloalkyl, and/or a haloalkyl;
(XXXV) a —SR, wherein R is selected from the group comprising a hydrogen, an alkyl, a cycloalkyl, or a haloalkyl;
(XXXVI) a nitro;
(XXXVII) a phosphoryl; or
a pharmaceutically acceptable salt thereof, or
(b) a therapeutically effective amount of a composition comprising a therapeutically effective amount of the compound of Formula (I), or a pharmaceutically acceptable salt thereof,
wherein the method comprises the binding of the compound of Formula (I), or a pharmaceutically acceptable salt thereof, to DNA of the human subject, and
wherein the pathological cellular processes are selected from the group consisting of replication, transcription, recombination, DNA repair, growth and development, cell differentiation, epigenetic regulation, regulation of DNA methylation, methyl transferase enzyme modulation, topoisomerase enzyme modulation, inhibiting of restriction enzyme digestion of DNA, inhibiting DNA dependent ATPase activity, inhibiting chromatin remodeling activity, and combinations thereof.

17. The method of claim 16, wherein the compound is of Formula (II): 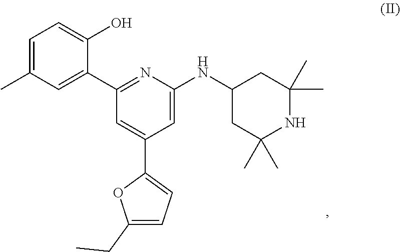
or a pharmaceutically acceptable salt thereof.

18. (canceled)

19. The method of claim 16, wherein the method comprises treating a disease, condition, or disorder selected from the group consisting of genetic diseases, epigenetic diseases, neurodevelopmental disorders, cardiac disorders, intellectual disabilities and developmental disorders, neurodegenerative diseases, immunological disorders, immune diseases, Huntington's disease and Fragile X Syndrome.

20. The method of claim 16, wherein the human subject suffers from a disease, condition, or disorder selected from the group consisting of genetic diseases, epigenetic diseases, neurodevelopmental disorders, cardiac disorders, intellectual disabilities and developmental disorders, neurodegenerative diseases, immunological disorders, immune diseases, Huntington's disease and Fragile X Syndrome.

21. A method for inhibiting ATP hydrolysis in the presence of DNA, or for inhibiting restriction digestion of DNA by an enzyme, the method comprising administering to a human subject in need thereof:
(a) a therapeutically effective amount of a compound of Formula (I): 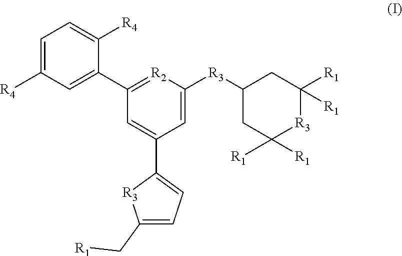
wherein R1 is independently selected from the group consisting of:
(I) hydrogen
(II) halogen
(III) alkyl or cycloalkyl;
(IV) alkenyl or cycloalkenyl;
(V) alkynyl or cycloalkynyl;
(VI) haloalkyl;
(VII) alkoxy;
(VIII) alkylamino;
wherein R2 is independently selected from the group consisting of:
(IX) a —N;
(X) a —P;
(XI) a —CR, wherein R is selected from the group comprising a hydrogen, an alkyl, a cycloalkyl, a haloalkyl, an alkylamino, or an alkoxy;
wherein R3 is independently selected from the group consisting of:
(XII) an —O;
(XIII) a —S;
(XIV) a —C(R)2, wherein R is selected from the group comprising a hydrogen, an alkyl, a cycloalkyl, a haloalkyl, an alkylamino, and/or an alkoxy;
(XV) a —NR, wherein R is selected from the group comprising a hydrogen, an alkyl, a cycloalkyl, or a haloalkyl;
(XVI) a —PR, wherein R is selected from the group comprising a hydrogen, a halogen, an alkyl, a cycloalkyl, or a haloalkyl; and
wherein R4 is independently selected from the group consisting of:
(XVII) hydrogen;
(XVIII) halogen
(XIX) alkyl or cycloalkyl;
(XX) alkenyl or cycloalkenyl;
(XXI) alkynyl or cycloalkynyl;
(XXII) haloalkyl;
(XXIII) aryl;
(XXIV) cyano;
(XXV) amino;
(XXVI) a —COOR, wherein R is a hydrogen or an alkyl;
(XXVII) a —OCOR, wherein R is an alkyl group;
(XXVIII) acyl;
(XXIX) sulfonyl;
(XXX) a —CON(R)2, wherein R is selected from the group comprising a hydrogen and/or an alkyl;
(XXXI) a —CONHR, wherein R is selected from the group comprising a hydrogen or an alkyl;
(XXXII) a —PO(OR)2, wherein R is selected from the group comprising a hydrogen and/or an alkyl;
(XXXIII) an —OR, wherein R is selected from the group comprising a hydrogen, an alkyl, a cycloalkyl, or a haloalkyl;
(XXXIV) an —N(R)2, wherein R is selected from the group comprising a hydrogen, an alkyl, a cycloalkyl, and/or a haloalkyl;
(XXXV) a —SR, wherein R is selected from the group comprising a hydrogen, an alkyl, a cycloalkyl, or a haloalkyl;
(XXXVI) a nitro;
(XXXVII) a phosphoryl; or
a pharmaceutically acceptable salt thereof, or
(b) a therapeutically effective amount of a composition comprising a therapeutically effective amount of the compound of Formula (I), or a pharmaceutically acceptable salt thereof,
wherein the method comprises the binding of the compound of Formula (I), or a pharmaceutically acceptable salt thereof, to DNA of the human subject, wherein the human subject suffers from a disease, condition, or disorder selected from the group consisting of genetic diseases, epigenetic diseases, neurodevelopmental disorders, cardiac disorders, intellectual disabilities and developmental disorders, neurodegenerative diseases, immunological disorders, immune diseases, Huntington's disease and Fragile X Syndrome.

22. The method of claim 21, wherein the method comprises inhibiting ATP hydrolysis in the presence of DNA.

23. The method of claim 21, wherein the method comprises inhibiting restriction digestion of DNA by an enzyme.
